(19) United States
(12) Patent Application Publication
(10) Pub. No.: US 2009/0312618 A1
(43) Pub. Date: Dec. 17, 2009

(54) ENDOSCOPIC DEVICE WITH BIOCHIP SENSOR

(76) Inventors: Arne Hengerer, Erlangen (DE); Rainer Kuth, Hochstadt (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/295,062

(22) PCT Filed: Mar. 27, 2007
(86) PCT No.: PCT/EP07/52898

§ 371 Date: Sep. 29, 2008

(30) Foreign Application Priority Data
Mar. 30, 2006 (DE) 10 2006 014 885.1

(51) Int. Cl. A61B 5/05; A61B 6/00

(52) U.S. Cl. 600/345; 600/476

(57) ABSTRACT

A device for performing minimally invasive procedures in vivo has an endoscope body that carries at least one sensor or actuator and at least one magnetic element allowing the body to be freely navigated in the body of a patient by an extracorporeally applied magnetic field. the body carries at least one biochip sensor with capture molecules for detection of biological molecules in samples. A processing and analyzing unit in the body is connected to the biochip sensor for optical or electrical evaluation of the samples.

### BACKGROUND OF THE INVENTION

1. Field of the Invention

The invention concerns a device to implement a minimally-invasive procedure inside the body of a patient, with at least one sensor and/or actuator, wherein the device has at least one magnetic element and the device is freely navigable inside the body by means of a magnetic coil system arranged outside the body of the patient.

2. Description of the Prior Art and Related Subject Matter

In medicine it is frequently necessary to implement a medical measure (for example a diagnosis or a treatment) internally, thus in the body of a (normally living) person or animal as a patient. The target area of such a medical measure is often a hollow organ in the appertaining patient, in particular the gastrointestinal tract. For a long time the medical measures have been conducted with the use of endoscopes which are inserted into the patient from the outside through body orifices of the patient or through small incisions in a non-invasive or minimally-invasive manner and are controlled or, respectively, positioned mechanically. Inspection or manipulation devices (for example a camera or a grabber) to execute a desired action are hereby located at the tip of a more or less long, flexible catheter. Additional devices can be slid into a working channel of the catheter to the tip and be retracted again from there. Conventional endoscopes exhibit various disadvantages; for example, they cause pain or even injury in the patient due to the indirect force transfer to the catheter tip upon feeding it in, due to its length and due to the friction effects that occur. Internal organs further away from the introductions can also be reached only with difficulty, or not at all.

Therefore, for a few years video capsules that the patient swallows have been known for catheter-free or tube-free endoscopy. The video capsule moves through the alimentary canal of the patient due to peristalsis and acquires a series of video images. These are transmitted extracorporeally and are stored in a recorder. The alignment of the capsule (and therefore the viewing direction of the video images) as well as the residence time in the body of the patient are random. Outside of image acquisition, the capsule has no active functionality. Diagnosis functions (such as targeted observation, cleaning, biopsies) are likewise just as impossible as targeted treatments inside the patient. A targeted diagnosis or finding cannot be implemented with this technology.

An endoscopy capsule that is equipped with a magnet and that can move by remote control by interaction with a gradient field generated by an external magnet system is known from DE 101 42 253 C1.

A magnetic coil system that is required in order to move the magnetic endoscopy capsule through hollow organs of a patient by means of magnetic (non-contact) force transfer is described in detail in DE 103 40 925 B3. The force transfer thus ensues in a targeted manner, without contact and with external control. These endoscopy capsules (also called endorobots) have the functionalities of a conventional endoscope (for example video acquisition, biopsy, medicine administration etc.). A medical procedure can be implemented autarchically (i.e. wirelessly or without catheter) with such an endorobot.

In German application 10 2005 032 368 (not previously published), an endorobot is lo described that is connected with a highly-flexible hose and that draws this hose behind it on its path through the hollow organs, with which treatment tasks (such as, for example, the supply of liquid or gaseous operating or working resources) can ensue, or which can be used for power supply.

The invention concerns a device of the aforementioned type.

Biotechnology and gene technology have acquired increasing importance in recent years. One basic object in biotechnology and gene technology is the detection of biological molecules such as DNA (deoxyribonucleic acid) or RNA (ribonucleic acid), proteins, polypeptides. In particular, molecules in which genetic information is encoded are of great interest for many medical applications. Through their detection, disease pathogens can be detected, for example in a blood sample of a patient. This makes the diagnosis significantly easier for the physician. Genetic tests can also facilitate the search for an optimal treatment in the treatment of tumor illnesses. While such examinations have previously been conducted in large laboratories, in the future biochip sensors will additionally refine and accelerate the diagnosis technology and displace it to the practice of the treating physician or to the household of the patient. Biochip sensors are generally known as very small sample substrates made from glass, plastic or silicon on which hundreds or thousands of biochemical reactions can run simultaneously. An evaluation of these reactions on the biochip sensor is also already possible today. With regard to their functionality, in particular their evaluation methods, biochip sensors can be roughly sub-divided into optical and electrical sensors, although the basic idea of all biochip sensors is identical. For example, substances known as probes or capture molecules are applied and immobilized on the sample substrate via by photo-lithographic methods or microdispersion, i.e. they are permanently affixed to the surface of the biochip sensor. If a sample to be examined is now brought into effective contact with the immobilized capture molecules, molecules complementary to the samples hybridize, meaning that specific molecules are bound to the samples according the hybridization rules. The bound molecules are identified through various detection methods. In one method in widespread use, the bonds are made visible by a fluorescent dye. In other detection methods, for example in which it is desired to detect a specific, known molecule, the bound molecules are charged with enzymes, for example, that cause a splitting or conversion of specific molecules and therefore contribute to changes of measurement variables (for example change of the pH value).

Certain carcinomas, in particular cancers of the large intestine, are presently able to be diagnosed only in a relatively late stage. Non-polyposis neoplasms occurring in the intestinal lumen are very difficult to diagnose with established methods. Most recently, imaging methods for in vivo diagnosis of molecular alterations have been added to generally known in vitro methods. New reagents and methods have been developed that effectively diagnose illness-associated molecular alterations in vivo. The developed, modern imaging techniques and the contrast agents (thus the molecular probes) interacting with specific molecular target structures form the basis of this technique. In the future, the development of molecular probes will enable neoplastic variation to be detected in an early stage of tumor genesis in which the transition from degenerate and reversible states can, however, be fluid. An invasive removal of early neoplasms must therefore be balanced against the risk of an intervention. Although minimally-invasive methods are suitable this purpose, since a resection can be conducted with almost no risk of a complication, unnecessary procedures are nevertheless always to be avoided. The correct point in time of an intervention is therefore to be determined. This is possible only to a limited extent with the aforementioned methods; a monitoring of the development after very early detection would be desirable.

It is known that various patients also show different reactions to medicines that are to be ascribed to cited genetic predispositions. A technique is accordingly also sought for the development of new therapeutic agents and for routine monitoring of pharmacological therapies that enables the monitoring of biochemical changes in the body, even over a longer time span.

In the prior art, colon carcinomas are detected by endoscopy of the large intestine by means of flexible endoscopes, wherein this is insufficient since only anatomically pronounced pathologies can be detected. The aforementioned imaging methods (for example by means of MR) can theoretically also detect molecular (and therefore early) changes; however, such approaches are purely diagnostic and do not enable resection in the same work step. At the same time, such methods are expensive due to the use of expensive apparatuses and cannot be applied comprehensively as a preventative (screening) examination. Economically reasonable prevention is thus not possible.

DE 10 2005 006 877 discloses a system and a method in which molecular probes (for example a tumor-specific contrast agent) are administered that enable an identification of a tumor. Various treatment methods (for example phototherapy, sonic therapy among others) are simultaneously proposed. The detection and treatment of molecular variations in a very early stage, including the possibility of the monitoring over a longer time span, is not discussed in this document.

### SUMMARY OF THE INVENTION

An object of the present invention is to provide a device that allows a comprehensive and location-dependent molecular diagnosis and intervention inside the body to be offered relatively inexpensively. It is also an object of the invention to monitor the effectiveness of specific pharmacological therapies and molecular diagnoses.

The object is achieved by a device of the aforementioned type that has at least one biochip sensor with capture molecules for detection of biological molecules in samples, and at least one preparation and analysis unit arranged within the device for optical or electrical evaluation of the samples is associated with said biochip sensor. The biochip sensor will advantageously be localized in the head region (i.e. at the tip) of the device that is movable inside the body. This endoscopic device enables a relatively cost-effective examination by the treating physician without expensive, high-technology medicine. The biochip sensor can be positioned well along a hollow organ, depending on the design of the device. The biochip sensor with its preparation and analysis unit is therefore brought directly to the location of the pathology, and a comprehensive test can be conducted. The acquired information is thus very specific information regarding the location of the pathology (for example receptor status, antigen expression, enzyme activities etc.). Relatively fast, comprehensive molecular examinations can be conducted via by the integrated biochip sensor. In this context, it is important that multiple parameters to be evaluated can now be detected in parallel. These examinations can be repeated relatively cheaply in order to conduct the collection of data even over a longer time span. If necessary, therapy can also be administered in the same work step via an intervention unit. The employed biochip sensor should be used to detect biological molecules and thereby be immobilized with capture molecules that hybridize according to the key-lock principle. Such capture molecules can represent oligonucleotides, proteins, polypeptides or polysaccharides. Alternatively, synthetic binding molecules (affibodies), viruses or bacteria can be applied as capture molecules. Furthermore, at least one preparation and analysis unit that advantageously stands in direct spatial coupling with the biochip sensor is associated with the biochip sensor. Either optical evaluations or electrical evaluations of the samples are possible by the preparation and analysis unit. For an optical detection method, the unit includes a number of mechanical and fluidic components for preparation and analysis of the samples in a typical manner. These components provide of the transport of the liquid samples, either via pumps or via the capillary effect in the thin channels. These samples are mixed with reagents via fluidic interfaces, for example they are marked with a fluorescent dye or with reagents for a bioluminescent reaction, and are supplied to the biochip sensor. The binding of specific molecules to the biochip sensor is made visible by means of the fluorescent dye. Residual dye is removed from the sample in multiple washing steps if necessary. The preparation and analysis unit likewise has the necessary optical exposure and evaluation unit. It is also conceivable that a preparation and analysis unit can embody multiple biochip sensors, and that the activation of the reaction surfaces of the biochip sensors can then be realized via microfluidic systems and valve controllers. For example, in such a case multiple biochip sensors can be arranged in an array next to the preparation and analysis unit. In other variants, the preparation and analysis unit has electrical measurement systems. In such measurement systems, changes of (normally physical or chemical) variables can be detected and analyzed. An example is the measurement of the change of the pH value on the chip surface, and represented as a change of the electrical potential difference before administration of a reagent (enzyme) and after its administration. Other methods detect mass changes using what are known as micro-scales by detecting the changing resonance frequencies with accumulation of substances at the capture molecules. Methods are also known that are based on interaction between an evanescent field occurring at the substrate surface and the molecules that bind to the substrate surface. Other methods in turn measure the change of the electrical charge of the biochip sensor.

A fast evaluation of samples even during the invasion can be advantageously enabled by the integration of the preparation and analysis unit into the head of the endoscopic device. This allows it to react directly to the sample results. If such a device as an endoscopy capsule is freely navigable in a hollow organ inside the body by externally controlled magnetic fields, the advantages of the invention are particularly enhanced. Such a capsule can execute the previously described analyses relatively independently without particularly stressing the patient.

In a preferred embodiment, the contacting of the capture molecules with the biological molecules to be examined or detected can be controlled by a covering element. According to the invention, a mechanical covering element is provided that should prevent an immediate contacting of the sensitive surface of the biochip sensor. In this way it is ensured that a precise positioning of the endoscopic device is possible in advance, and that a contamination of the capture molecules occurs only at the desired position. In addition to rigid a mechanical covering element, a covering element can be used that independently triggers in the body after a certain time period and in this way enables the contact possibility of the surface of the biochip sensor.

In another embodiment, the control of the covering element (and therefore the control of the contacting of the capture molecules) ensues externally, i.e. from outside the body. The covering element should thereby be externally manipulable, such that an uncovering of the surface of the biochip sensor is possible. Such a manipulation or even control of the covering element can ensue relatively simply by external magnetic fields or radio-controlled switching devices. In other cases, the manipulation of the covering element can also ensue by the emission of specific substances that result in a dissolution of the covering element. In an additional case, for example, the covering element can be exposed to a simple, externally monitored and controlled heat treatment so as to unblock the contacting of the biochips based on this treatment.

In a further embodiment of the invention, the device has a magazine that contains a number of biochip sensors with the same characteristics of the capture molecules. It is known that biochip sensors can have on their surface multiple hundreds of sites known as spots or positions with a diameter of approximately 100 μm; each spot encompassing a large number of capture molecules, and a test is respectively completed in this spot. For the implementation of multiple test series (in particular if these should be implemented over a longer time span) it is desirable to have multiple, identical biochip sensors available, i.e. multiple biochip sensors with identical characteristics of the immobilized capture molecules. According to the invention, these biochip sensors can be arranged in a magazine (i.e. a storage container) and are able to be individually transformed from there into a contact, measurement or analysis position. The magazine and the arrangement of the biochip sensors in the magazine can be assigned in a number of ways. In one embodiment, for example, the biochip sensors are arranged in a stacked arrangement, similar to a weapon magazine; in another arrangement, the biochip sensors can be in sequence on a flexible substrate, similar to a feed device of components for an automatic population system. The number of the biochip sensors arranged in the magazine is set up according to the use case and the size of the chips. In an additional development, portions of the preparation and analysis unit can be connected with the biochip sensor as necessary and likewise be accommodated in the magazine. In this way it is possible to acquire comparative measurement series. In this embodiment of the invention, the device should have multiple biochip sensors within this storage magazine (which biochip sensors are identical in their characteristics), and the feed of the biochip sensors into the analysis position ensues either automatically under time control or can again be externally controllable. Using the proposed magazine technique it is moreover possible to adapt the biochip sensors with regard to their size to the proposed use in the body, in particular to the use in a hollow organ. For example, just as many tests can be completed with two or more biochip sensors each with a smaller surface as on one chip of larger surface, and by means of a serial arrangement of the chips an arrangement is achieved that corresponds to the natural longitudinal extent of the hollow organ, and thus is matched to this use case.

In an additional embodiment, the device has a magazine that contains a number of biochip sensors with different characteristics of the capture molecules. The magazine itself is identical to the aforementioned embodiments. This variant is advantageously used where a larger number of the most varied tests should be enabled. Since the chip surface is always immobilized with specific capture molecules, and therefore only a limited number of substances can be tested, it is advantageous to populate the aforementioned magazine as proposed. This applies all the more if a reduction of the substrate surface is necessary due to the limited space requirement for the application inside the body, in particular inside a hollow organ. In this case, an arrangement in the longitudinal direction of the device mobile in the body is appropriate.

In another embodiment of the invention, the device has at least one repository in which chemical or biological substances can be stored. Substances or reagents are in particular required in the preparation and analysis unit, such as given preparation of a sample through a lysis or in the analysis of the sample itself via enzyme administration, for example. Water, which can likewise be provided by such repositories, is also required for some preparation steps.

In one variant, the substances stored in the repository can also be discharged into the body. This can be particularly advantageous when, for example, the location of the examination is still not entirely determined, or it is not certain whether a closer examination should occur at all. Namely, in some cases the endoscopic device according to the invention should initially be used with a specific molecular probe, i.e. a contrast agent to mark specific points (possibly already points of suspicion) in order to detect neoplasms, for example. In this case, an optically fluorescing substance is preferred. In other cases, the contrast agent can already have been administered otherwise in order to mark large regions. In this case, however, a re-administration at specific locations via the endoscopic device can support the detailed diagnosis. The administration of the substances ensues either via injection needles of an injection unit or via outlet channels on the shell of the device that are in the position to emit substances.

The control of this injection unit should likewise advantageously be possible from the outside. For example, this can ensue through externally controllable magnetic fields, or using radio-controlled release devices or (in the simplest case) by mechanical release devices. The fine positioning of the endoscopic device normally ensues visually via the known optical sensors of these devices. A monitored administration of the substances is possible in this way.

In an additional variant of the invention, the device has a flexible supply channel that is directed to the outside and in this way represents an external connection of the endoscopic device mobile inside the body. The possibility of any sort of supply exists via this supply channel, which is designed for the particular case and can have multiple sub-channels. Supplying light, supplying energy or even supplying with biological or chemical substances or with water are thus possible. Not every supply channel needs to support all supply types; rather, an adaptation to the specific supply case is possible.

In a preferred embodiment, the supply channel is connected with the repository, and this can therefore be supplied via the supply channel. The repositories are thus refillable. For cases where there must be no contamination of substances due to transport in the same supply channel, the supply channel will advantageously have multiple supply tubes in parallel, separated from one another.

According to another exemplary embodiment, the supply channel is connected with the device such that a direct discharge of biological or chemical substances inside the body is also possible via the channel. This is particularly advantageous when, for example, molecular probes (thus contrast agent) should be discharged that, as mentioned above, should enable marking, for example of neoplasms. The advantage of this embodiment is that the required quantity of contrast agent is always available and can be administered in a targeted manner.

In an additional preferred embodiment of the invention, light of an excitation wavelength for luminescence excitation of the molecule probes can be injected via the supply channel, and this light enables a surface illumination of a hollow organ. The administered contrast agent is excited by the injection of light of a specific wavelength and begins to fluoresce. For this purpose, the entire hollow organ (for example the intestine) must be exposed with light of the excitation wavelength. For this purpose the supply channel has at least one optical conductor, and the external end of the optical conductor is connected with an external laser source. The endoscopic device is designed such that light radiation onto the wall of the hollow organ is achieved by transparent light emission apertures. The light emission apertures can be distributed over the entire circumferential of the shell of the device and thus ensure a 360° light radiation. In another case, a single light radiation beam moving forward or back can be sufficient. The illumination effect will normally be supported by an overlapped movement of the device in the hollow organ.

In an associated embodiment of the invention, the device has a stereo-optic system. An accumulation of the molecular probes in the pathological intestinal wall is detected and localized by this stereo-optic system, which advantageously is achieved with CCD cameras. The images are transferred out and shown on a display unit. The detection of pathological variations can ensue with software support by means of suitable image recognition software, or the acquired images are merely presented on the display unit for monitoring by the physician. A software-supported evaluation can already ensue inside the body; at least a pre-processing would be possible here. The endoscopic device is moved forward along the hollow organ for optical scanning. If an accumulation of contrast agent is detected, the precise position of the pathological location can be determined via the position of the camera sensor itself and be sent out, for example via radio. Neoplastic changes can thus already be diagnosed in a very early stage in this manner.

In an additional embodiment, the device has a tissue extractor. This is particularly appropriate if a pathological conspicuity has been diagnosed and localized based on the aforementioned examinations. The tissue extractor can be an integrated biopsy device (for example a capillary tube) that is injected into the location of the previously-localized pathological conspicuity in order to extract the tissue sample. The actuation of the biopsy device can ensue by means of a propellant unit that provides the required force transfer, for example.

The extracted tissue sample is now further analyzed by the integrated biochip sensor. For this purpose, the capillary tube of the biopsy device is supplied via a suitable interface to the preparation and analysis unit, which then makes the sample available to a biochip sensor. Known optical or electrical analysis methods are thereby generally applied.

In an additional variant, the device has at least one intervention unit that is based on a laser radiation or thermal radiation source. An intervention unit of such a design can be used in order to treat a previously detected pathological alteration whose pathological finding has been bioanalytically confirmed. The power supply for such intervention units ensues inductively or via the supply channel, for example. The intestine can therefore be remediated via the intervention unit in a minimally-invasive manner in patients with increased cancer risk of the colon. Neoplastic degenerations are detected in the early stage and are preventatively removed in a targeted manner, wherein the application naturally is not limited to the intestinal region.

The endoscopy capsule according to the invention can be provided with an anchor device that holds the capsule in the tissue of the patient, so the capsule is held in position even without the action of the external magnetic field. In such a case, it would even be conceivable for the patient to be merely relocated in an external magnetic coil system in order to reposition the capsule. In this case, multiple patients could be treated in parallel without continuously requiring the magnetic coil system.

### BRIEF DESCRIPTION OF THE DRAWINGS

FIG. 1 schematically illustrates an endoscopic device constructed and operating in accordance with the present invention in the form of an endoscopy capsule, in a side view.

FIG. 2 illustrates the endoscopic device of FIG. 1, with a supply channel.

FIG. 3 schematically illustrates the endoscopic device shown in FIG. 2, in a view rotated by 900 relative to the view of FIG. 2.

FIG. 4 schematically illustrates a magazine for biochip sensors in accordance with the invention, in a side view.

FIG. 5 schematically illustrates the magazine of FIG. 4, in an end view with the covering element in place.

FIG. 6 shows the end view of the magazine of FIG. 5, with the covering element displaced.

FIG. 7 schematically illustrates a further embodiment of the magazine for biochip sensors in accordance with the present invention.

### DESCRIPTION OF THE PREFERRED EMBODIMENTS

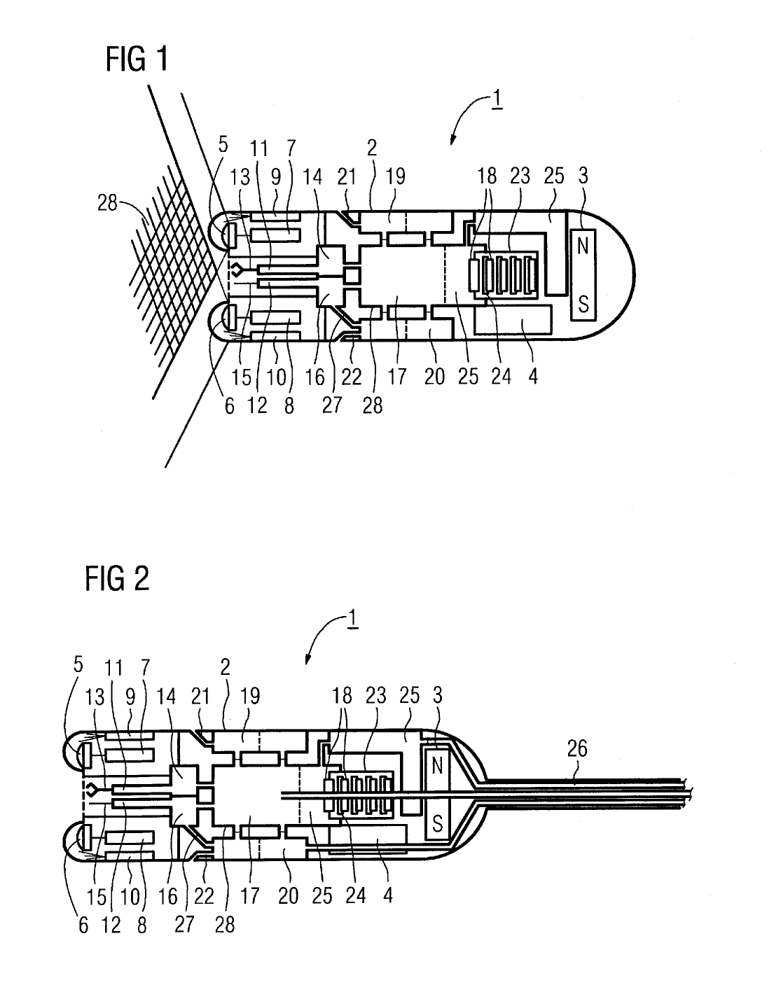

FIG. 1 shows the endoscopy device according to the invention in the form of an endoscopy capsule magnetically navigable via an external magnetic field. The device 1 is fitted into the housing 2, in which a magnetic element 3 is integrated. The magnetic element 3 is, for example, a permanent magnet, a weakly magnetic element magnetizable in a magnetic field or an electronic coil. The magnetic element 3 interacts with navigation magnetic fields that are generated via an external magnetic coil system (not shown) so that the device 1 admitted into the patient body can be externally controlled and moved. A control device 4 in the form of a microcontroller is also integrated into the oblong, cylindrical housing 2 exhibiting a diameter of approximately 10 mm to 15 mm. The control device 4 takes over all control tasks pertaining to the function devices of the endoscopic device 1, which are described in detail in the following. Data lines from and to the control device 4 were omitted for reasons of clarity. The endoscopic device 1 movable in the body has two cameras 5, 6 with acquisition direction in the longitudinal direction of the device 1. The cameras are spaced from one another far enough that stereoscopic acquisitions are possible. The cameras 5, 6 are connected with the transmission units 7, 8. The transmission units are in the position to send the images acquired by the cameras 5, 6 to a receiver (not shown in detail) of the endoscopic device outside of the body via an antenna (not shown). The illumination devices 9, 10 (which are advantageously equipped with LEDs) enable the illumination of the exposure field of the camera 5, 6. A biopsy device 11 and an injection device 12 are located between the cameras 5, 6. The biopsy device 11 has a grabber 13 that can be triggered via an actuator device 14 and thus is in the position to engage with the tissue of the patient. Using the grabber 13 it is possible to bring about an anchoring of the endoscopic device 1. It is also possible to extract possible tissue samples from the inner wall of the hollow organ to be examined. The biopsy device 11 is triggered by the control unit 4. The injection device 12 is located separate from the biopsy device 11 and operates in its own channel. The injection device 12 has an injection needle 15 that is in a position to penetrate into the tissue of the patient if necessary and there conduct injections. For this purpose, the injection device 12 is connected via a channel 27 with a repository 19, 20. The injection device 12 is likewise able to take in bodily fluids directly from the tissue if necessary. For this purpose, the injection needle does not necessarily have to leave its housing channel, but nevertheless may be configured to do so. The acquired fluids can be supplied via a channel 28 to a preparation and analysis unit 17. The preparation and analysis unit 17 is in the position to prepare the extracted sample so that it can be supplied to a biochip sensor 18. The preparation and analysis unit 17 is also connected with the repositories 19, 20. Biological or chemical substances that are necessary to preparation and analyze the sample are located in these repositories. The repositories 19, 20 also directly connected to the outside via the channels 21, 22; the endoscopic device 1 is thus in the position to discharge specific biological or chemical substances into the body of the patient via the channels 21, 22. According to the exemplary embodiment presented in FIG. 1, multiple biochip sensors are stored in a magazine 23. The biochip sensors 18 are inactive due to the present of a covering element 24 and are activated upon removal of the covering element 24. The evaluation of the biochip sensor 18 ensues in the analysis region 25 of the preparation and analysis unit 17. For this purpose, the analysis region 25 can be emptied into the consumable materials tank 28 and cleaned.

FIG. 2 shows that the repositories 19 and 20 as well as the consumable materials tank 28 can be connected to an external supply channel 26 in an optional embodiment. The repositories 19 and 20 can be supplied with liquid substances via this channel 26. A transport of contaminated substances away from the consumable materials tank 28 is also possible. For this the supply channel 26 has various supply sub-channels separated from one another.

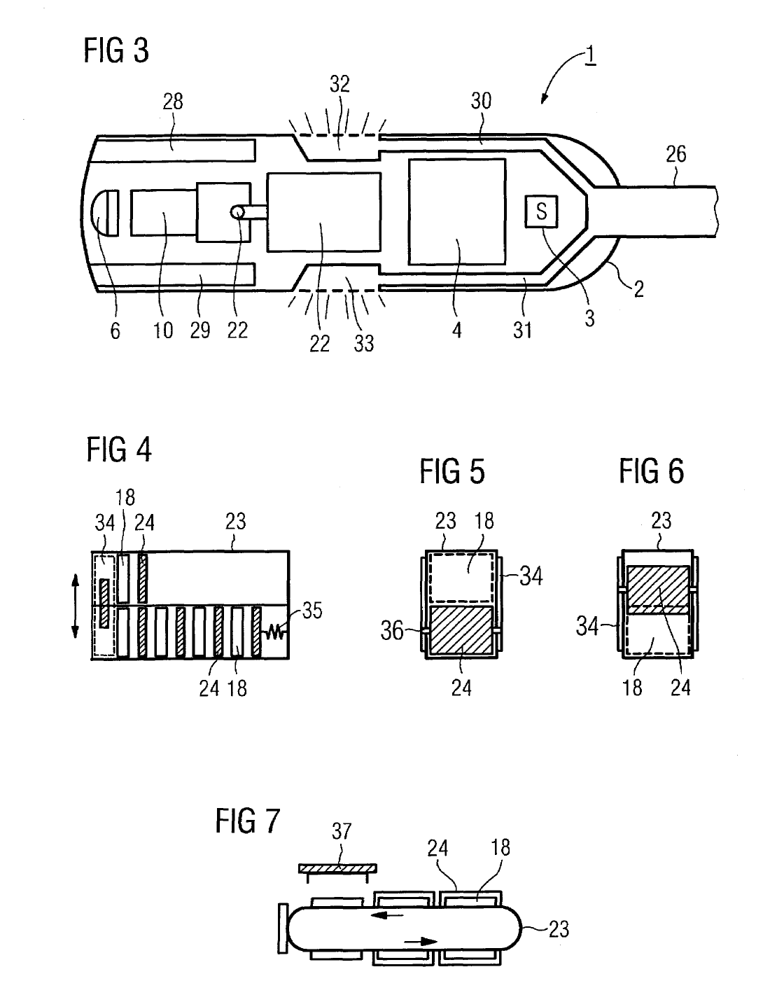

FIG. 3 shows a principle drawing of the endoscopic device 1 presented in FIG. 2 in a side view, wherein the device 1 has been rotated by 90° clockwise along the longitudinal axis relative to FIG. 2. For clarity, FIG. 3 shows only the function units situated in the upper image layer. The intervention units 29 and 38 shown above and below the second image acquisition unit 6 and the illumination device 10 associated with it serve for the treatment of possible neoplastic degenerations. For this purpose, the intervention unit units 29 and 38 can form a radiation source via which laser radiation or thermal radiation can be applied. The supply channel 26 shown in FIG. 3 is connected via optical conductor channels 30, 31 with illumination regions 32, 33. The housing wall of the housing 2 is designed to be transparent in the illumination regions 32, 33 so that the light which is injected via the supply channel 26 enables an areal illumination of the hollow organ in which the endoscopic device is located. If light of a specific excitation wavelength is injected via the supply channel 26, a luminescence excitation of the molecular probes previously discharged by the discharge channels 21, 22 is possible as necessary.

FIG. 4, FIG. 5 and FIG. 6 each show a principle drawing of a magazine to provide biochip sensors. FIG. 4 shows the magazine 23 in side view. The magazine 23 is divided into a lower magazine region and an upper magazine region. The unused biochip sensors 18 that are respectively covered by the covering element 24 are located in the lower magazine region. The already-used biochip sensors 18 and the removed covering element 24 are stored in the upper magazine region. The magazine 23 is completely closed except for one side. An entrainment unit 34 via which both the covering element 24 and the biochip sensors 18 can be moved from the lower magazine region into the upper magazine region is located at the open side. The transport of the covering element 24 and the biochip sensors 18 in the lower magazine region in the direction of the magazine opening is ensured by the spring device 30. The entrainment unit 34 is ideally attached to the outer wall of the housing of the magazine 23. The engagement of the entrainment unit 34 in the covering element 24 and the biochip sensors 18 can ensue via catches 36 that, for example, engage as noses in the corresponding recesses in the covering element 24 and the biochip sensors 18.

FIG. 5 shows the front view of the opening of the magazine 23. The covering element 24 shown hatched there is located in the engagement and should be displaced into the upper region of the magazine 23 via the entrainment unit 34.

FIG. 6 shows the displacement of the covering element 24 into the upper region of the magazine 23. The underlying biochip sensor 18 is uncovered in this manner. After using this sensor, it can likewise be slid into the upper region of the magazine.

FIG. 7 shows a principle drawing of an additional embodiment of the magazine 23 to provide biochip sensors 18. According to FIG. 7, the magazine 23 is presented in the form of a revolving band. The biochip sensors 18 are attached to this. Each of the sensors 18 is covered or packaged by a covering element 24. The biochip sensor 18 currently in use is located on the facing side of the revolving magazine 23. Before it reaches this position, the covering element 24 is removed by the uncovering unit 37 shown here. The uncovering unit 37 can in this case be an electromagnet which is controllable and thus can cover the correspondingly designed covering element 24.

Although modifications and changes may be suggested by those skilled in the art, it is the intention of the inventors to embody within the patent warranted heron all changes and modifications as reasonably and properly come within the scope of their contribution to the art.

1-17. (canceled)

18. A device for implementing a minimally-invasive procedure in vivo, comprising:
a device body configured to be freely movable inside the body of the patient;
a magnet carried by the device body allowing navigation of the device body inside the body of the patient by interaction with an extracorporeally generated magnetic field; and
at least one biochip sensor carried by said device body, said at least one biochip sensor comprising capture molecules that detect biological molecules in samples, and at least one preparation and analysis unit in said device body, in communication with said at least one biochip sensor, for optical or electrical evaluation of said sample captured by said biochip sensor.

19. A device as claimed in claim 18 comprising a retractable covering element that controls contact of said capture modules with said biological molecules.

20. A device as claimed in claim 19 comprising an external control unit in communication with said covering element that controls covering and uncovering of said capture molecules by said covering element.

21. A device as claimed in claim 19 comprising a magazine containing a plurality of biochip sensors each having capture molecules with identical characteristics, said covering element covering one end of said magazine.

22. A device as claimed in claim 19 comprising a magazine containing a plurality of biochip sensors each having capture molecules with different characteristics, said covering element covering one end of said magazine.

23. A device as claimed in claim 18 comprising a repository in said device body for storing chemical or biological substances.

24. A device as claimed in claim 23 comprising a discharge element in communication with said repository that allows discharge of the substance in said repository into the body of the patient.

25. A device as claimed in claim 24 comprising an external control unit that controls discharge of said substance from said repository into the body of the patient.

26. A device as claimed in claim 1 comprising a flexible supply channel connected to said device body.

27. A device as claimed in claim 26 wherein said preparation and analysis unit consumes electrical power, and wherein said supply channel comprises an electrical line to supply said electrical power to said preparation and analysis unit.

28. A device as claimed in claim 26 wherein said supply channel comprises a conduit allowing supply of a substance to the device body for discharge from the device body into the body of the patient.

29. A device as claimed in claim 10 wherein said supply channel comprises a waveguide allowing light at an excitation wavelength to be supplied to the device body for radiation from the device body to illuminate an area of the body of the patient outside of the device body.

30. A device as claimed in claim 29 comprising a stereo-optic unit allowing an accumulation of biological or chemical substances to be localized.

31. A device as claimed in claim 18 comprising a tissue extractor carried by said device body for interaction with tissue surrounding said device body in the body of the patient.

32. A device as claimed in claim 18 wherein said biochip sensor comprises capture molecules that allow a tissue analysis to be conducted on tissue in the body of the patient adjacent to the device body.

33. A device as claimed in claim 18 comprising a radiator, selected from the group consisting of laser radiators and thermal radiators, that emits an interventional radiation beam from the device body into the body of the patient.

34. A device as claimed in claim 18 comprising a fastening device that anchors the device body in tissue in the body of the patient.
